(19) United States
(12) Patent Application Publication
(10) Pub. No.: US 2013/0149317 A1
(43) Pub. Date: Jun. 13, 2013

(54) MALARIA VACCINE

(71) Applicants: Takafumi Tsuboi, Matsuyama-shi (JP); Motomi Torii, Toon-shi (JP); Tatsuya Sawasaki, Matsuyama-shi (JP); Yaeta Endo, Matsuyama-shi (JP)

(72) Inventors: Takafumi Tsuboi, Matsuyama-shi (JP); Motomi Torii, Toon-shi (JP); Tatsuya Sawasaki, Matsuyama-shi (JP); Yaeta Endo, Matsuyama-shi (JP)

(73) Assignee: CELLFREE SCIENCES CO., LTD., Yokohama-shi, Kanagawa (JP)

(21) Appl. No.: 13/809,462

(22) PCT Filed: Jul. 4, 2011
(86) PCT No.: PCT/JP2011/065258

§ 371 Date: Jan. 10, 2013

(30) Foreign Application Priority Data
Jul. 21, 2010 (JP) 2010-164228

(51) Int. Cl. A61K 39/015; A61K 39/395

(52) U.S. Cl. CPC A61K 39/015; A61K 39/39575
USPC 424/172.1; 424/191.1; 424/130.1

(57) ABSTRACT

The present invention relates to a malaria vaccine comprising:

(a) a polypeptide consisting of an amino acid sequence of SEQ ID NO: 1, 2, or 3;

(b) a polypeptide consisting of an amino acid sequence of SEQ ID NO: 1, 2, or 3, wherein one or more amino acids are deleted, substituted and/or added and having effect for preventing falciparum malaria; or

(c) a polypeptide consisting of an amino acid sequence having 70% or more identity with an amino acid sequence of SEQ ID NO: 1, 2, or 3 and having effect for preventing falciparum malaria.

### TECHNICAL FIELD

The present invention relates to a malaria vaccine.

### BACKGROUND ART

Malaria is widely spread in tropical and subtropical regions. Malaria is caused by infection with malaria parasites mediated by anopheles. Of four kinds of human malaria, falciparum and vivax malaria account for the majority of them. Both cause symptoms, such as fever and anemia. Falciparum malaria causes death if accompanied by serious complications. After World War II, the number of deaths caused by malaria was reduced by measures against mediating mosquitoes using insecticides such as DDT and the appearance of a specific medicine, chloroquine. However, as chloroquine-resistant Plasmodium falciparum and insecticide-resistant mosquitoes subsequently emerged, the number of patients increased again. Currently, about 300 million people are affected by falciparum malaria, causing estimated deaths of more than 860,000 every year. Thus, malaria vaccines have attracted attention as new specific medicines.

However, malaria parasites express vastly different genes depending on the developmental stages of their complicated life cycles. Hence, three types of malaria vaccines have been investigated: (1) vaccines to prevent the infection targeting to sporozoites and liver-stage parasites, (2) vaccines to prevent the developing the disease targeting to erythrocyte-stage parasites and (3) vaccines to prevent the spreading of parasites in the mosquito gut. However, none has been put to practical use. Thus, the development of malaria vaccines is awaited.

### Disclosure of Invention

### Problems to be Resolved by the Invention

The objective of the present invention is providing malaria vaccine.

### Means of Solving the Problems

Malaria vaccines have been investigated using limited candidate molecules, which have attracted attention for decades, to be put to practical use. Of these vaccines, those to prevent infection using a certain surface protein of sporozoite, injected from a mosquito into the human body, as an antigen have most extensively been investigated. A phase II clinical trial was completed with a response rate of about 50%. However, the results of the phase II clinical trial demonstrated that the effects of the vaccines were insufficient in themselves.

In October 2007, “malaria eradication,” which had remained undeclared for many years, was declared again to the world, emphasizing the importance of developing new malaria vaccines as a priority issue. Candidates, more potent than previous vaccine molecules, have been explored. It has long been known that inhabitants in endemic regions carry protective antibodies to inhibit the growth of erythrocyte-stage parasites and that protective immunity is induced when experimentally immunized with irradiated sporozoites (so to speak, a live vaccine against parasites). Overall immune responses against parasites lead to various protective effects. Specifically, comprehensively exploring malaria parasite molecules, involved in these immune responses, may lead to the development of multivalent vaccines comprising multiple malaria parasite antigens.

The malaria genome project estimated the presence of about 5,400 genes in P. falciparum. About 60% of these genes were demonstrated to be functionally unknown in 2002. The data were published on the malaria parasite genome database (PlasmoDB: http:llplasmodb.org/plasmo/). At this time, new candidate antigens for malaria vaccines were identified one after another. Thus, many researchers expected that research on malaria vaccines would be dramatically facilitated.

However, to utilize the genome database for exploring candidate vaccine antigens, recombinant proteins should be synthesized. The genome-wide expression of P. falciparum genes was attempted using an Escherichia coli system in the United States and Europe. One thousand genes were expressed. However, only 6-21% of them were synthesized as soluble proteins. Furthermore, from the viewpoint of protein folding, recombinant proteins are preferably synthesized in a eukaryotic cell system, instead of an E. coli system.

A unique method utilizing a wheat germ protein synthesis system to produce recombinant proteins in vitro was turned into actual utilization by Ehime University.

This synthesis method, derived from eukaryotic cells of wheat, was actually more suitable for expressing the recombinant proteins of human, mice and plants than an E. coli system. In addition, a cell-free system imposes no restrictions associated with a living cell system, such as cytotoxicity of synthesized proteins. Hence, the cell-free system should be suitable for producing the recombinant proteins of malaria parasite, a eukaryotic cell pathogen.

Thus, 567 genes were selected from the P. falciparum genome database. Of these, 478 (84%) genes were successfully expressed using the wheat cell-free system. Of these, 26 molecules expressed during the erythrocyte stage were selected as vaccine candidates to inhibit the onset of disease. Following the synthesis and purification of recombinant proteins using the wheat germ cell-free protein synthesis system, antibodies were raised against them and two polypeptides that antibodies against them inhibited the growth of cultured P. falciparum strain were identified and thereby the present invention is completed.

More specifically, the present invention is as follows:

[1] A malaria vaccine comprising:
(a) a polypeptide consisting of an amino acid sequence of SEQ ID NO: 1, 2, or 3;
(b) a polypeptide consisting of an amino acid sequence of SEQ ID NO: 1, 2, or 3, wherein one or more amino acids are deleted, substituted and/or added and having effect for preventing falciparum malaria; or
(c) a polypeptide consisting of an amino acid sequence having 70% or more identity with an amino acid sequence of SEQ ID NO: 1, 2, or 3 and having effect for preventing falciparum malaria.
[2] A malaria vaccine according to [1], wherein the polypeptide was synthesized by a wheat germ cell-free protein synthesis method.
[3] A malaria vaccine according to [1] or [2], further comprising an antibody involved in the sialic acid-dependent pathway.
[4] A malaria vaccine according to [3], wherein the antibody involved in the sialic acid-dependent pathway is an anti-EBA-175 antibody.
[5] A method for preventing falciparum malaria, comprising administrating a malaria vaccine according to any one of [1]-[4] to a subject in need such treatment.

### b Effect of the Invention

The malaria vaccine of the invention is useful for preventing falciparum malaria.

### BRIEF DESCRIPTION OF THE DRAWINGS

FIG. 1 is SDS-PAGE for the synthesis and purification of recombinant proteins.

FIG. 2 is antibody reactivity (indirect fluorescent antibody technique).

FIG. 3 shows vaccine effects.

FIG. 4 shows binding to the erythrocyte surface.

FIG. 5 shows the additive inhibitory effects of anti-GAMA and anti-ESA-175 antibodies on the growth of P. falciparum.

### BEST MODE FOR CARRYING OUT THE INVENTION

The polypeptide of the present invention can be obtained by expressing the polynucleotide encoding the polypeptide. A nucleic acid comprising the polynucleotide of the present invention may be in a form of either single or double strand. The double-stranded polynucleotide of the present invention may be inserted into an expression vector to prepare a recombinant expression vector in order to express the protein of the invention. Specifically, the nucleic acids of the present invention also include a recombinant expression vector, prepared by inserting the double-stranded polynucleotide of the present invention into an expression vector.

The “protein comprising the amino acid sequence wherein one or more amino acids are deleted, substituted and/or added” of the present invention refers to artificially-modified polypeptides or proteins, such as allelic mutants present in vivo.

The number and positions of amino acid mutations in the polypeptide of the present invention are not limited as long as the activity of the polypeptide of the present invention is maintained. Thus, the number and positions of amino acid residues to be deleted, substituted and/or added without inactivation can be determined using a computer program well known to those skilled in the art. For example, the percentage of mutations is typically 10% or less and preferably 5% or less of total amino acids. To maintain a protein conformation, amino acids are preferably substituted with those having the same properties, such as polarity, charge, solubility, hydrophobicity, amphiphilicity and hydrophilicity, as the ones to be substituted.

Amino acid sequence identity as used herein is about 70% or more, preferably about 80% or more, more preferably about 90% or more and most preferably about 95% or more.

The term “sequence identity” as used herein refers to identity between two polypeptide sequences. The “sequence identity” is determined by comparing two sequences optimally aligned over a sequence region to be compared. In this context, the proteins to be compared may have an addition or a deletion (e.g., gap) in the optimally-aligned sequences. Such sequence identity may be calculated by preparing an alignment using, for example, Clustal W algorithm with Vector NTI (Nucleic Acid Res., 22(22): 4673-4680(1994)).

Expression vectors used herein may be optionally selected depending on the hosts to be used, purposes and the like, and include plasmids, phage vectors and viral vectors.

For example, vectors used for Escherichia coli hosts include plasmid vectors, e.g., pUC118, pUC119, pBR322 and pCR3 and phage vectors, e.g., λZAPII and λgt11. Vectors used for yeast hosts include pYES2 and pYEUra3. Vectors used for insect cell hosts include pAcSGHisNT-A. Vectors used for animal cell hosts include plasmid vectors, e.g., pCEP4, pKCR, pCDM8, pGL2, pcDNA3.1, pRc/RSV and pRc/CMV and viral vectors, e.g., retroviral, adenoviral and adeno-associated virus vectors.

The above vectors may optionally contain elements, such as inducible promoter, signal sequence, selection marker and terminator. To facilitate isolation and purification, a sequence may be added to allow the expression of a fusion protein with thioredoxin, His tag, GST (glutathione S-transferase), or the like. For this purpose, GST fusion protein vectors having a suitable promoter that functions in a host cell (lac, tac, trc, trp, CMV, SV40 early promoter, etc.), such as pGEX4T, vectors having a tag sequence (Myc and His, etc.), such as pcDNA3.1/Myc-His and a vector expressing a fusion protein with thioredoxin and His tag (pET32a) may be employed.

The above expression vector may be used to transform a host to generate a transformant containing the expression vector. Hosts used herein include Escherichia coli, yeast, insect cells and animal cells. Escherichia coli strains include E. coil K-12 lines, such as HB I01, C600, JM109, DH5α and AD494 (DE3) strains. Yeasts include Saccharomyces cerevisiae and Pichia pastoris. Animal cells include L929, BALB/c3T3, C127, CHO, COS, Vero, Hela and 293-EBNA cells. Insect cells include sf9.

An expression vector may be introduced into host cells using a conventional method suitable for the above host cells. Specifically, it may be carried out with calcium phosphate method, DEAE-dextran method, electroporation, or the like. Following the introduction, the cells are cultured in a conventional medium containing a selection marker, thus allowing the selection of transformants containing the expression vector.

The protein of the present invention may be produced by culturing the transformants under appropriate conditions. The resultant protein may be further isolated and purified according to standard biochemical procedures. In this context, purification procedures include salting out, ion exchange chromatography, absorption chromatography, affinity chromatography and gel filtration chromatography. The protein of the present invention, expressed as a fusion protein with thioredoxin, His tag, GST, or the like as described above, can be isolated and purified by purification procedures using the properties of such fusion protein or tags.

Nucleic acids comprising polynucleotides encoding the peptide of the present invention fall within the scope of the nucleic acid of the present invention.

The polynucleotide encoding the polypeptide of the present invention may be in a form of either DNA or RNA. The polynucleotide of the present invention can be easily prepared based on the amino acid sequence of the peptide of the invention or DNA encoding the same. Specifically, it can be prepared by conventional methods, such as DNA synthesis and PCR amplification.

A malaria vaccine containing the polypeptide of the present invention as an active ingredient may be administered in a mixture with or in combination with a pharmaceutically acceptable carrier.

Administration methods include intradermal, subcutaneous, intramuscular and intravenous administration. The dose of the polypeptide of the present invention in formulation is appropriately adjusted depending on a disease to be treated and patient's age, weight and the like, and preferably ranges from 0.0001 to 1,000 mg, preferably from 0.001 to 1,000 mg, and more preferably from 0.1 to 10 mg once for several days or months.

### EXAMPLE 1

PF08_{—}0008 (PlasmoDB gene code: PF08_{—}0008 (http://plasmodb.org/)) is one of proteins whose expression is expected during the merozoite stage when P. falciparum invades erythrocytes. PF08_{—}0008 is also referred to as GPI-anchored micronemal antigen (GAMA) (Eukaryotic Cell, Dec. 2009, 1869-1879), which binds to erythrocyte surface at the C-terminal region in a sialic acid-independent manner. The full-length sequence (SEQ ID NO: 1) used herein to express a recombinant protein was obtained by PCR amplification using a merozoite-stage cDNA template from cultured P. falciparum 3D7 strain (MR4: Malaria Research and Reference Reagent Resource Center (http://www.mr4.org/)).

MAL7P1.119 (PlasmoDB gene code: MAL7P1.119 (http://plasmodb.org/)) is a protein whose expression is expected during the merozoite stage when P. falciparum invades erythrocytes. A partial fragment of 239-amino acid sequence of the present protein (hereinafter referred to as Fragment_{—}4 (SEQ ID NO: 2)) used to express a recombinant protein in the present invention was obtained by PCR amplification using a merozoite-stage cDNA template from cultured P. falciparum 3D7 strain.

A target gene was cloned into the Xhol/NotI site of the multiple cloning site of pEU-E01-GST-TEV-MCS-N2 which is a plasmid obtained by introducing GST and TEV into pEU-E01-MCS-N2 (CellFree Sciences) for wheat germ cell-free protein synthesis system.

Conditions of Expression:

Transcription was carried out in 1.2 ml volume at 37° C. for 6 hours using pEU-E01-GST-TEV-N2 vector, into which cDNA of the full-length PF08_{—}0008 or MAL7P 1.119 Fragment 4 was inserted, as a template. A total amount of the mRNA obtained was added to 1.2 ml of wheat germ cell-free protein synthesis kit WEPRO (TM) 1240G (240 OD/m1) (CellFree Sciences) and dispensed into all the wells of a 6-well plate to carry out protein synthesis by the double layer method at 17° C. for 16 hours.

Purification of Antigen:

The protein synthesis reaction solution obtained (28.8 ml) was mixed with 300 μl of Glutathione Sepharose 4B (GE Health Care), followed by adsorption at 4° C. for 16 hours. The resin was transferred into a column and washed. Then, 300 μl of PBS containing 1.2 units of TEV protease was added for cleavage reaction at 30° C. for 3 hours to obtain purified protein.

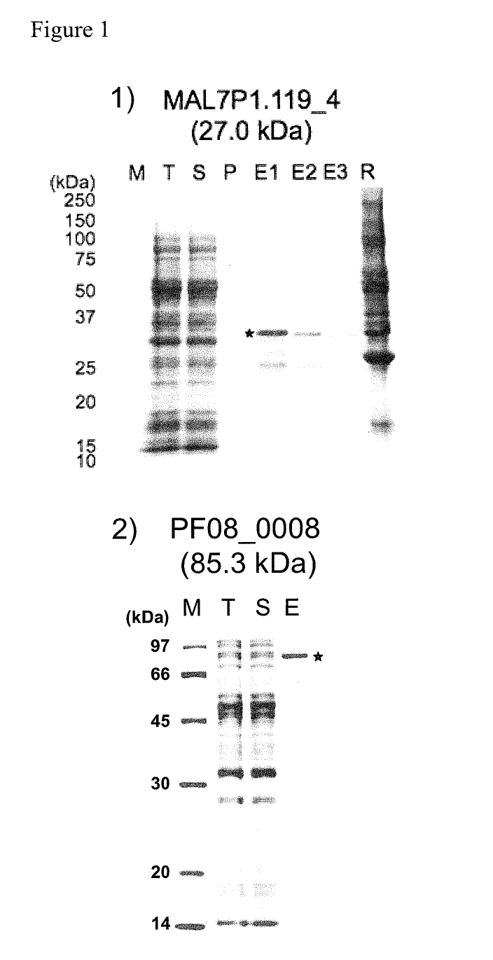

MAL7P 1.119 Fragment_{—}4 was synthesized and purified as a band slightly larger than the expected molecular size. The full-length recombinant protein of PF08_{—}0008 was synthesized and purified at the expected size (FIG. 1).

Immune Processing of Antigen:

To obtain an antiserum against PF08_{—}0008 or MAL7P1.119 Fragment_{—}4, the purified recombinant protein, adjusted to the concentration of 0.25 mg/0.4 ml PBS, was emulsified with 400 μl of Freund's complete adjuvant (Wako Pure Chemical Industries, Ltd.) to be administered subcutaneously at multiple sites in the back of a female white

Japanese rabbit (KBL, KITAYAMA LABES CO., LTD.). The negative control group using one rabbit per each group was immunized in the same manner with GST prepared similarly in a cell-free protein synthesis system. At 3 weeks after the initial immunization, the rabbits were boosted with Freund's incomplete adjuvant (Wako Pure Chemical Industries, Ltd.), followed by booster immunization twice in total at 3-week interval. At 2 weeks after the last immunization, whole blood was collected from the carotid artery under anesthesia with pentobarbital sodium. The collected blood was allowed to stand at room temperature for 1 hour and then at 4° C. overnight, followed by serum separation on the following day. The separated serum was stored frozen at −80° C. until use in the experiment.

Validation of Antibody Reactivity to Parasites:

To observe the reactivity of the antiserum prepared against parasites using a confocal laser microscope, cultured P. falciparum strain 3D7 was spotted onto a glass slide and fixed with acetone, and subsequently, the slide was incubated with the above anti-rabbit antiserum as a primary antibody at 37° C. for 1 hour and then with anti-rabbit IgG Alexa488 conjugate (Invitrogen) as a secondary antibody at 37° C. for 30 minutes, and after washing, the slide was sealed using an antifade (ProLong Gold Antifade Reagent, Invitrogen) in PBS and observed with a confocal laser microscopy.

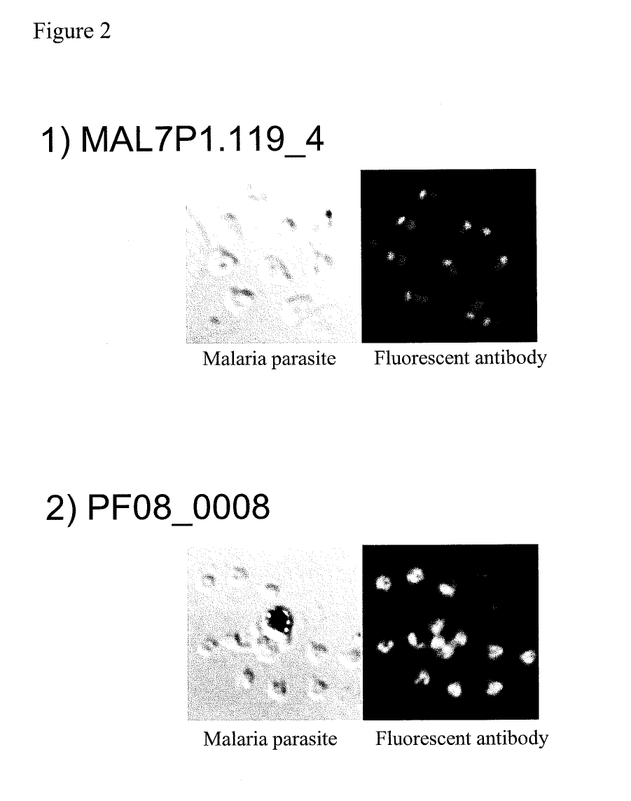

The rabbit antiserum against MAL7P1.119 Fragment_{—}4 reacted with the apical organelle, which is considered to play an important role in the invasion of P. falciparum merozoites into erythrocytes. The rabbit antiserum against PF08_{—}0008 also reacted with the apical organelle of P. falciparum merozoite (FIG. 2).

Determination of Vaccine Effects:

To examine the vaccine effects of a rabbit antiserum against PF08_{—}0008 or MAL7P1.119 Fragment_{—}4, an IgG fraction purified from the rabbit antiserum through a protein G column was added to cultured P. falciparum strain 3D7 to determine inhibition rates on parasite growth without IgG addition ({1-LDH absorbance of parasite with IgG addition/LDH absorbance of parasite without IgG addition}×100).

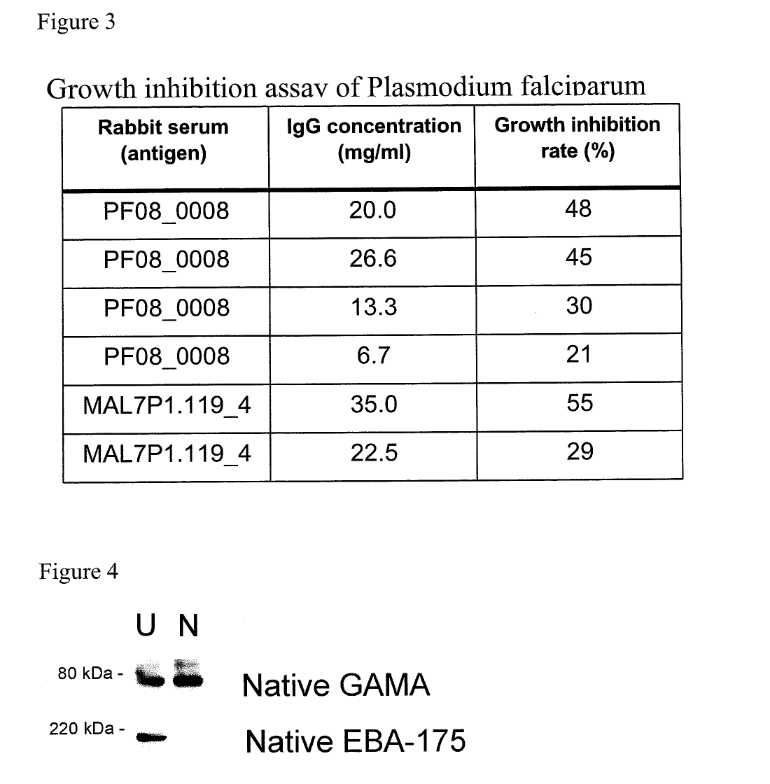

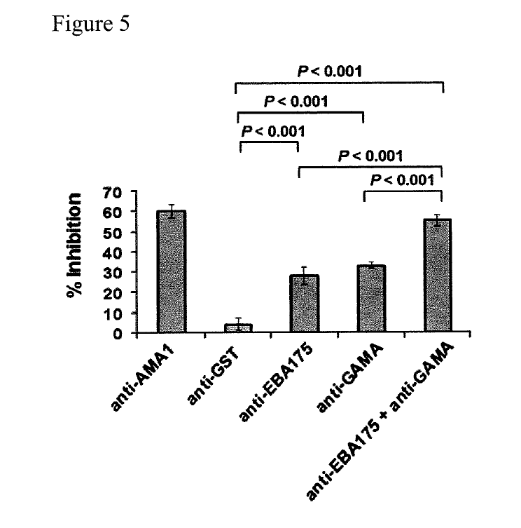

The inhibition rate of anti-PF08_{—}0008 rabbit IgG on the growth of P. falciparum was enhanced by 21-45% in a concentration-dependent manner when the IgG concentration in the culture medium of parasite was increased stepwise from 6.7 to 26.6 mg/ml. In another experiment, the inhibition rate was 48% at an IgG concentration of 20.0 mg/ml. The inhibition rate of anti-MAL7P 1.119 Fragment_{—}4 rabbit IgG on the growth of P. falciparum was 29% when the IgG concentration in the culture medium of parasite was 22.5 mg/ml. The inhibition rate was 55% when the IgG concentration was increased to 35.0 mg/ml (FIG. 3).

Thus, the two P. falciparum proteins, PF08_{—}0008 and MAL7P1.119 Fragment_{—}4 were considered to be useful as the vaccine antigens of falciparum malaria.

### EXAMPLE 2

A polypeptide, synthesized for PF08_{—}0008 with the N-terminal signal sequence and the C-terminal GPI anchor signal sequence removed, i.e., the ecto-domain from N at position 25 to A at position 714 (ecto-domain: SEQ ID NO: 3), was used to immunize a rabbit. As a result, the inhibition rate of anti-rabbit IgG PF08_{—}0008 ecto-domain rabbit IgG on the growth of P. falciparum was 50% when the IgG concentration in the culture medium of parasite was 35.0 mg/ml.

Thus, the PF08_{—}0008 ecto-domain was considered to be useful as the vaccine antigen of falciparum malaria.

### EXAMPLE 3

GAMA and the C-terminal fragment of GAMA (Tr3: 500-714 of GAMA) bind to the erythrocyte surface.

A full-length GAMA protein (native GAMA) from cultured P. falciparum binds to normal erythrocytes (U) and neuraminidase-treated erythrocytes (N: sialic acid removed). When the same experiment was conducted using EBA-175 derived from parasites, which is known to bind to erythrocytes via sialic acid, EBA-175, unlike GAMA, did not bind to neuraminidase-treated erythrocytes (N). As described above, the followings were demonstrated: (1) GAMA binds to erythrocyte surface; (2) the binding domain is present at the C-terminal, aa500-714; and (3) binding is sialic acid-independent. Thus, synergistic or additive effects on the inhibition of parasite invasion are expected when an anti-GAMA antibody involved in the sialic acid-independent pathway and an anti-EBA-175 antibody involved in the sialic acid-dependent pathway simultaneously act on the invasion of P. falciparum into erythrocytes.

The inhibitory effects on the growth of P. falciparum were additively enhanced when the anti-GAMA and anti-EBA-175 antibodies coexist, as compared with each antibody alone.

IgG purified from rabbit antiserum were added to cultured P. falciparum in vitro at the concentrations below to compare inhibitory effects on parasite growth. Inhibition rates are as follows: (1) 60% for anti-AMA1 IgG (final concentration 20 mg/ml) in the positive control group, whose growth-inhibiting activity is well known, (2) 4% for anti-GST IgG (final concentration 20 mg/ml) in the negative control group, (3) 28% for the simultaneous addition of anti-EBA-175 (final concentration 4 mg/ml) and anti-GST (final concentration 16 mg/ml) antibodies, (4) 33% for the simultaneous addition of anti-GAMA IgG (final concentration 16 mg/ml) and anti-GST (final concentration 4 mg/ml), and (5) 55% for the simultaneous addition of anti-EBA175 (final concentration 4 mg/ml) and anti-GAMA (final concentration 16 mg/ml) antibodies.

Thus, the vaccine effects can be enhanced when an anti-GAMA antibody involved in the sialic acid-independent pathway and an anti-EBA-175 antibody involved in the sialic acid-dependent pathway simultaneously act on the invasion of P. falciparum into erythrocytes.

### INDUSTRIAL APPLICABILITY

The malaria vaccine of the present invention is useful for the prevention of falciparum malaria.

1. A malaria vaccine comprising:
(a) a polypeptide consisting of an amino acid sequence of SEQ ID NO: 1, 2, or 3;
(b) a polypeptide consisting of an amino acid sequence of SEQ ID NO: 1, 2, or 3, wherein one or more amino acids are deleted, substituted and/or added and having effect for preventing falciparum malaria; or
(c) a polypeptide consisting of an amino acid sequence having 70% or more identity with an amino acid sequence of SEQ ID NO: 1, 2, or 3 and having effect for preventing falciparum malaria.

2. A malaria vaccine according to claim 1, wherein the polypeptide was synthesized by a wheat germ cell-free protein synthesis method.

3. A malaria vaccine according to claim 1 or 2, further comprising an antibody involved in the sialic acid-dependent pathway.

4. A malaria vaccine according to claim 3, wherein the antibody involved in the sialic acid-dependent pathway is an anti-EBA-175 antibody.

5. A method for preventing falciparum malaria, comprising administrating a malaria vaccine according to claim 1 to a subject in need such treatment.


SEQUENCE LISTING (3 sequences)

SEQ ID NO: 1 - protein, 738 aa, Plasmodium falciparum
MKYYTSLYVALIIALCQAVSALIRNSNTPQAFLIPELNNNEKNEFNNNEKNEMNNNLNNEFNNNEENCDIQKIAEEMMENLLNENDMYTNIMLSLQNRLSDDYLCSEPKYENICIHEKDKISLSFPCSNPKYEKLIHKFTFKKLCNSKAAFNNTLLKSFIEEDEEQNTFSLMLKQFKILLTCVDDELKDIYKESIDLLVDLKTSITELTQKLWSGKMVNVLKKREFLITGILCELRNGNKSPLISKSLEFENLGILKMNNEELLNEAYNAFSDYYYFFPYFIQKLLEKGGMIERLIKIHENLTKYRTKDMVNKINAQSKGEVLNNEDILNKLNAYKHYTKHGATSFIQSREVKIVNQNVNNDDTTKNQQQNVNNNEKLNNNNNNNNNQQVNNNNNNNNQQVNNNNNNNNNQVNNNNNNNNNQVNNNNYNNNNQVNNNNNNNQQVNNNNNYNNQLNNNNFNNNLQVNKNDKHVPKNNHTTATHTNNILYNPLYSINPEKPKDIIKLLKDLIKYLHIVKFENNEPTTNIDEEGIRKLLENSFFDLNDDILIVRLLLKPQTVILTVIQSFMLMTPSPSRDAKAYCKKALINDQLVPTNDTNILSEENELVNNFSTKYVLIYEKMKLQELKEMEESKLKMKYSKTNLSALQVTNPQNNKDKNDASNKNNNPNNSSTPLIAVVTDLSGEKTEDIINNNVDIATLSVGVQNTFQGPNAKAGSLINHLSYATFLFFSFILINLLN

SEQ ID NO: 2 - protein, 239 aa, Plasmodium falciparum
NGKKDKNGVFVKLMNDQNDDGDDTKDGDDTKDEDDHKNEDDHKNEDDHKNEDDHKNEDDHKNGDDNKNGDDHKNGDDNKNGDDDNGKKSHDISDIKNIIDTILQSDDITDEQKKYLEIIKKILDLEEDVLNKEKEQLQLNKNIIEVLMGKSDELRNIAVNLKNGNGDNESSQRVDLAQNIVSNLLNFSVQLKNTGNIVYNNIQGQGELLQSIEKNIDKAENDLKKSTSVNTTFTPKNVP

SEQ ID NO: 3 - protein, 690 aa, Plasmodium falciparum
NSNTPQAFLIPELNNNEKNEFNNNEKNEMNNNLNNEFNNNEENCDIQKIAEEMMENLLNENDMYTNIMLSLQNRLSDDYLCSEPKYENICIHEKDKISLSFPCSNPKYEKLIHKFTFKKLCNSKAAFNNTLLKSFIEEDEEQNTFSLMLKQFKILLTCVDDELKDIYKESIDLLVDLKTSITELTQKLWSGKMVNVLKKREFLITGILCELRNGNKSPLISKSLEFENLGILKMNNEELLNEAYNAFSDYYYFFPYFIQKLLEKGGMIERLIKIHENLTKYRTKDMVNKINAQSKGEVLNNEDILNKLNAYKHYTKHGATSFIQSREVKIVNQNVNNDDTTKNQQQNVNNNEKLNNNNNNNNNQQVNNNNNNNNQQVNNNNNNNNNQVNNNNNNNNNQVNNNNYNNNNQVNNNNNNNQQVNNNNNYNNQLNNNNFNNNLQVNKNDKHVPKNNHTTATHTNNILYNPLYSINPEKPKDIIKLLKDLIKYLHIVKFENNEPTTNIDEEGIRKLLENSFFDLNDDILIVRLLLKPQTVILTVIQSFMLMTPSPSRDAKAYCKKALINDQLVPTNDTNILSEENELVNNFSTKYVLIYEKMKLQELKEMEESKLKMKYSKTNLSALQVTNPQNNKDKNDASNKNNNPNNSSTPLIAVVTDLSGEKTEDIINNNVDIATLSVGVQNTFQGPNAKA